(19) United States
(12) Patent Application Publication
(10) Pub. No.: US 2018/0206773 A1
(43) Pub. Date: Jul. 26, 2018

(54) DYNAMIC ASSESSMENT AND REHABILITATION SYSTEM FOR VERTIGO PATIENTS AND THE APPLICATION METHOD THEREOF

(71) Applicant: NATIONAL YANG-MING UNIVERSITY, TAIPEI CITY (TW)

(72) Inventors: Chung-Huang YU, TAIPEI CITY (TW); Po-Yin CHEN, TAIPEI CITY (TW); Li-Wei CHOU, TAIPEI CITY (TW); Chung-Lan KAO, TAIPEI CITY (TW); Yi-Shun CHUNG, TAIPEI CITY (TW)

(21) Appl. No.: 15/691,048

(22) Filed: Aug. 30, 2017

(30) Foreign Application Priority Data
Jan. 26, 2017 (TW) 106103236

(51) Int. Cl. A61B 5/00; A61B 5/11

(52) U.S. Cl. CPC A61B 5/4023; A61B 5/6803; A61B 5/742; A61B 5/1128; A61B 5/1121; A61B 5/0024; A61B 2505/09; A61B 5/7455; A61B 5/112; A61B 2560/0431; A61B 2562/0219; A61B 2562/0223; A61B 5/7405

(57) ABSTRACT

The present application provides a dynamic assessment and rehabilitation system for vertigo patients and the application method thereof, which uses a moveable monitoring platform to monitor and test a walking user and coordinates a first wearing unit to observe the head-turning condition to ensure the operating intensity of the user, in additional, by disposing a terminal module capable of recording the using condition of the user and analyzing and calculating the signals generated during the test process. In accordance with aforesaid system, the physiologic status of a vertigo patient can be assessed in a walking status and the patient can be given a feedback corresponding thereto, therefore raising the using efficiency of the user.

### FIELD OF THE INVENTION

The present invention relates to an assessment and rehabilitation system and the application method thereof, in particular, relates to a dynamic assessment and rehabilitation system for vertigo patients and the application method thereof.

### BACKGROUND OF THE INVENTION

Vertigo relates to a sort of sensory disorder resulted from delusion of movement and distortion of gravity direction. When vertigo occurs, a patient may have a feeling that himself or herself, or the surrounding environment is rotating, it is because of the incoordination between the patient's vision, proprioceptive feeling and sense of balance results in the patient being unable to maintain the space discrimination thereof and the pose balance of body thereof, and further influencing the body feeling and the movement ability thereof Vertigo may be resulted from many reasons and not more easily happened on any specific age group of human. However, when an old patient is having vertigo, he or her may have second injury resulted from limb collisions. In accordance with a statistical data, about 20% to 30% of people had vertigo experiences, currently it is still necessary to actively take precaution for vertigo.

Although there are many reasons causing vertigo, about more than 50% of the pathogenic causes are resulted from following three diseases: benign paroxysmal positional vertigo (BPPV), vestibular neuritis and Meniere's disorder, and the exception of the pathogenic causes has a certain extent relation with the imbalance of vestibular system. Accordingly, based upon the object of preventing and treating vertigo disease, the functional assessment of vestibular system of a patient, and the related rehabilitations after the vestibular system of the patient losing normal function, become several very important key points.

A vestibular system includes inner ears and a part of brain. The vestibular system is in charges of keeping balance of a human body in daily life. The activities of a human body is based on rotation and movement, accordingly the vestibular system achieve controlling the balance of rotation and lateral movement by using the semicircular canal system and the otoliths system, too. The semicircular canal system is in charge of sensing the change of angular acceleration of a head to perceive rotational action, and the otoliths system is in charge of finding out gravity, linear acceleration and the oblique of the head, therefore perceiving the linear-acceleration condition. The aforesaid information is contributed to the vestibular nuclei and cerebellum of a human body, and then transferred to the cerebral cortex and communicates the center and spinal cord managing eyeball activity of the brain stem, therefore causing corresponding eyeball rotation and body response for a human body keeping sight and trunk thereof stable. Accordingly, when the aforesaid parts of a human body are broken, have inflammation response, or incoordination or contradiction between different systems, the vertigo symptoms may be caused.

For the assessment of a vestibular system, currently the static dynamic visual acuity (DVA) test is a usual method. However, merely based on the static and simple test for the gazing function, it is unable to judge whether the recovered strength of the vestibular of a patient is enough to handle the activities of daily life, and the patient will not fall and have secondary collision. Additionally, the assessment method with insufficient activity data may cause the user losing his or her confidence. The user may lower or avoid the daily activities in accordance with being afraid of having vertigo symptoms or falling by losing the balance thereof, accordingly the function of the vestibular balance system further degenerates, and the user falls into a vicious circle that further refusing the daily activities.

In another aspect, currently several adapting or compensation motions including action control of eyeball, alternating head motion, walking around an obstacle or walking on a treadmill, have been used for the rehabilitation training of the vestibular system, however an assessment system is also needed for a user to realize the rehabilitation condition thereof Thus, how to design a dynamic assessment and rehabilitation system for vertigo patients, which provides better sensitivity and more matching the condition of the daily activities of a vertigo patient for the vertigo patient assessing and progressing rehabilitation training, has become an object to be urgently solved in the related art.

### SUMMARY OF THE INVENTION

A primary object of the present application is to provide a dynamic assessment and rehabilitation system for vertigo patients, by the design of the apparatus, a vertigo patient can execute assessment or rehabilitation under walking condition, therefore the system can provide accurate assessment content by the way more closing to the life situation of a user.

Another object of the present application is to provide a dynamic assessment and rehabilitation system for vertigo patients, by the design of the apparatus, an image signal or a locating signal can be extracted when a vertigo patient is operating the system, therefore providing the system enough information excepted to the head signal to judge the vertigo condition of the user.

Another object of the present application is to provide a dynamic assessment and rehabilitation system for vertigo patients, by the design of the apparatus, the system can immediately execute an assessment in accordance with the operation condition of the vertigo patient and adjust the rehabilitation schedule of the vertigo patient in accordance with the immediate feedback.

Another object of the present application is to provide a dynamic assessment and rehabilitation application method for vertigo patients, according to the method, a vertigo patient can execute tests related to functions of the vestibular system with a kept distance separated from the apparatus under a dynamic-walking operation, therefore the object executing tests under a dynamic-walking operation can be achieved.

Another object of the present application is to provide a dynamic assessment and rehabilitation application method for vertigo patients, according to the method, a vertigo patient can execute trainings related to functions of the vestibular system with a kept distance separated from the apparatus under a dynamic-walking operation, and execute an assessment both considering a surrounding-reaction training and a dynamic-walking signal, therefore the vertigo patient can have the most suitable training mode and the efficiency of rehabilitation can be enhanced.

To achieving aforesaid objects, the present application discloses a dynamic assessment and rehabilitation system for vertigo patients, including: a first wearing unit, a moveable monitoring platform and a terminal module; wherein the first wearing unit includes a first wearing main body ware on a head of a user and at least a sensor installed on the first wearing main body, wherein the sensor is configured to sense at least a sensing signal of the user and transfer the at least a sensing signal; additionally, the moveable monitoring platform includes a platform main body, a moving assistant assembly, a signal-receiving device, a distance sensor, at least a display device and a control module, wherein the platform main body includes a base and a monitoring machine arranged above the base, and the moving assistant assembly is installed below the base, and the signal-receiving device is arranged above the monitoring machine to receive an image signal generated from when a user is operating the system, and the distance sensor is arranged above the monitoring machine to measure a monitored distance between the user and the platform main body to receive a distance signal, and the at least a display device is arranged above the monitoring machine to provide at least a display information, and the control module is arranged above the platform main body and electrically connected to each of the signal-receiving device, the distance sensor, the moving assistant assembly and the at least a display device, wherein the control module receives and transfers the dynamic action signal, and the control module further receives and analyzes the distance signal to drive the moving assistant assembly to displace and controls a display information of the at least a display device to display in accordance with a first instruction information; finally, the terminal module is arranged at an any arranging point in the moveable monitoring platform or within a signal-reachable range, and the terminal module makes connections with the control module and the at least a sensor, and the terminal module receives the distance signal and the image signal transferred from the control module, and then executes analysis the signals before providing the first instruction information to the control module, and then receives the at least a sensing signal coordinating the dynamic action signal to execute an assessment of the physiological status of the user.

In an embodiment of the present application, this discloses that the dynamic assessment and rehabilitation system for vertigo patients further includes at least a second wearing unit. The second wearing unit includes a second wearing main body and a signal-emitting device, wherein the second wearing main body is ware on the trunk of the user, and the signal-emitting device is arranged above the second wearing main body to project a displacement signal to the signal-receiving device.

In an embodiment of the present application, this discloses that the displacement signal includes a light signal, a Wi-Fi signal or a Bluetooth signal.

In an embodiment of the present application, this discloses that the signal-emitting device can measure and transfer signal including: an angular speed signal, an acceleration signal, an angular acceleration signal, a sensing signal of magnetic field location or any combination thereof to the terminal module.

In an embodiment of the present application, this discloses that the base and the monitoring machine are formed in one piece to be connected or connected by a telescopic element, wherein the telescopic element stretches along an extended direction perpendicular to a ground surface.

In an embodiment of the present application, this discloses that the dynamic assessment and rehabilitation system for vertigo patients further includes a track, wherein the moveable monitoring platform is installed on the track to displace, and the patient executes assessment and rehabilitation in the track.

In an embodiment of the present application, this discloses that the dynamic assessment and rehabilitation system for vertigo patients further includes a training-guiding module is disclosed, the training-guiding module can be a loudspeaker device, an earpiece device, a vibrating motivation device, a light-touching motivation device, a stroking motivation device, a tapping motivation device or an air-flow motivation device.

In an embodiment of the present application, this discloses that the control module can be input with a second instruction information to drive the moving assistant assembly to displace and control a display information of the at least a display device.

In an embodiment of the present application, this discloses that the control module can generate a third instruction information after receiving the distance signal by analyzing the signal by itself, therefore driving the moving assistant assembly to displace and control a display information of the at least a display device.

In an embodiment of the present application, this discloses that the terminal module, the control module and the at least a sensor are electrically connected to each other or connected via wireless communication.

In an embodiment of the present application, this discloses that the sensor signal includes: an inclined or rotated angle signal of a head, an angular speed signal, an acceleration signal, an angular acceleration signal, a location signal of a head or any combination thereof.

In an embodiment of the present application, this discloses that the display information includes a text information, an image information or a virtual scenes information.

In additional, to achieve aforesaid objects, the present application disclosed an application method of the dynamic assessment and rehabilitation system for vertigo patients, configured to dynamically assess the physiologic status of a vertigo patient, including steps of: the user initiating walking, and the distance sensor receiving a distance signal of the user and the platform main body, and after being executed by a signal process step, the distance signal can inform the control module to drive the moveable monitoring platform to keep a monitoring distance with the user; the user watching the at least a display device and turn the head thereof, therefore the sensor sensing the at least a sensing signal and transferring that to the terminal module; the terminal module judging the at least sensing signal reaching a first threshold value before transferring the first instruction to the control module to control the display information displayed on the at least a display device; the user responding a reaction result in accordance with the display information of the at least a display device, and transferring the reaction result to the terminal module; alternating above steps until the information gathered by the terminal module is enough to assess the physiologic status of the user.

In an embodiment of the present application, this discloses that before the step of the user initiates walking, further arranging the moveable monitoring platform facing the user wearing the first wearing unit and keeping the monitoring distance between the moveable monitoring platform and the user.

In an embodiment of the present application, this discloses that the signal-receiving device receives an image signal formed by an operation the user operates the system, and recording the image signal by transferring the image signal to the terminal module via the control module.

In an embodiment of the present application, this discloses that the user wears the at least a second wearing unit, and when the user is walking the second wearing unit can project a displacement signal to the signal-receiving device, and the displacement signal is transferred to the terminal module via the control module to be recorded in the terminal module.

In an embodiment of the present application, this discloses that the first threshold value is the angular speed of the head turning of the user being between 30 degrees/second and 550 degrees/second, or is the head displacement being between 10 cm and 50 cm, or is the extent of the turning angle being between 15 degree and 60 degree or any combination thereof.

In additional, to achieve aforesaid objects, the present application disclosed an application method of the dynamic assessment and rehabilitation system for vertigo patients, configured to provide rehabilitation trainings to a vertigo patient, including steps of: the user initiating walking, and the distance sensor receiving a distance signal of the user and the platform main body, and after being executed by a signal process step, the distance signal can inform the control module to drive the moveable monitoring platform to keep a monitoring distance with the user; the user watching the at least a display device and turn the head thereof, therefore the sensor sensing the at least a sensing signal and transferring that to the terminal module; the terminal module judging the at least sensing signal reaching a first threshold value before transferring the first instruction to the control module to control the display information displayed on the at least a display device; the user responding a reaction result in accordance with the display information of the at least a display device, and transferring the reaction result to the terminal module; the terminal module analyzing and judging in accordance with the sensor signal and the reaction result to generate an updated first instruction information to the control module, therefore controlling the display device displaying an updated display information for the user to keep training; alternating above steps until the operation condition of the user reach a target of the rehabilitation.

In an embodiment of the present application, this discloses that when the user is executing the step of turning the head thereof, the system further provides a training guiding module providing a prompt message to help the user execute rehabilitation trainings.

In an embodiment of the present application, this discloses that the prompted message can includes: a sound prompt, a voice prompt, a prompt of tone change, a text prompt, a prompt of twinkling frequency, a vibration prompt, a prompt of touching-free tactile, a prompt of light touch, a stroke prompt, a tapping prompt or any combination thereof.

### BRIEF DESCRIPTION OF THE DRAWINGS

FIG. 1: a side view diagram of a preferred embodiment of the present application;

FIG. 2: a side view diagram of another view angle of a preferred embodiment of the present application;

FIG. 3: a side view diagram of another preferred embodiment of the present application;

FIG. 4: a flow chart of a preferred embodiment of the application method of the present application;

FIG. 5: a flow chart of a preferred embodiment of the application method of the present application.

### DETAILED DESCRIPTION OF THE INVENTION

### Definitions

For helping the Examiner further realize and recognize the present application, some detailed descriptions and preferred embodiments are described as follows:

In the present application, a new dynamic assessment and rehabilitation system for vertigo patients and the application method thereof is provided. An assessing result closest to the life status of a user can be achieved by a way of allowing the user to execute walking in association with executing an functional assessment of vestibular, therefore an objective and accurate analysis can be done. In additional, a dynamic assessment and rehabilitation system for vertigo patients and the application method thereof of the present application can judge the rehabilitation status of a user by different reference date resulted from corresponding apparatus design thereof, and the training difficulty can be immediately adjusted to be close to the rehabilitation demand of the user, therefore the entirety of the rehabilitation efficiency and be raised.

Accordingly, the present application provides a dynamic assessment and rehabilitation system for vertigo patients, and the system includes: a first wearing unit capable of immediately measuring a data related to the head-turning of a user when the user is walking, and the related data can be a relative foundation for the system to judge whether the user reaches a threshold of a test. In additional, the present application provides a moveable monitoring platform capable of providing information to the user for testing vestibular functions and keeping a fixed distance with the user at the same time, besides, the moveable monitoring platform can achieve images resulted from the walking of the user to assess the rehabilitation status of the vertigo patient in a more pluralistic way. Finally, the present application provides a terminal module not only capable of gathering all information during an operation process of the user to generate a most suitable test information, but distributing and recording the information for the user or the treating staffs to realize all details of the rehabilitation process, therefore reaching the object of a dynamic assessment and rehabilitation system of the present application, including: achieving the assessment result closest to the life condition of the user, executing objective and accurate analysis and immediately and effectively adjusting the difficulty of the training, therefore the object of raising the entirety of the rehabilitation efficiency can be achieved.

Based on above orientations, some elements, features and the combination thereof and the actuated relation of each other thereof of an electric walking aids for promoting gait activities are further described below:

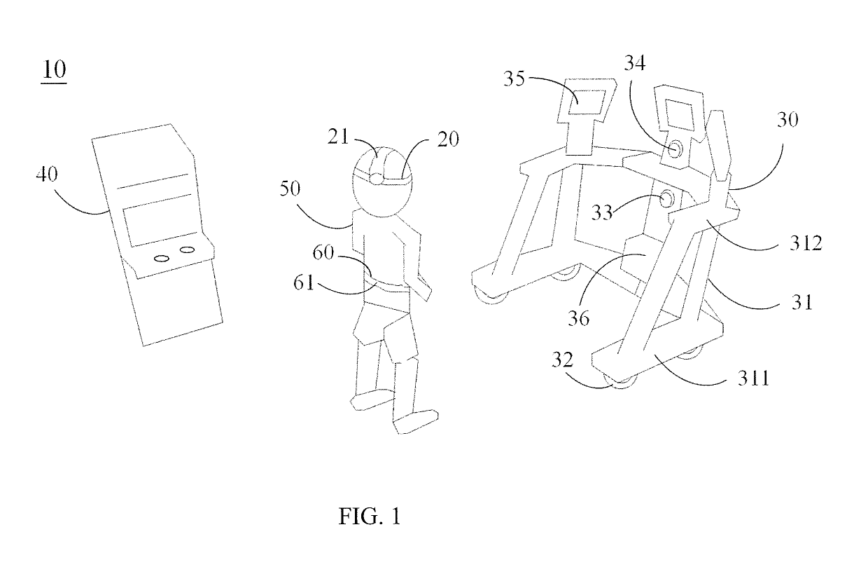

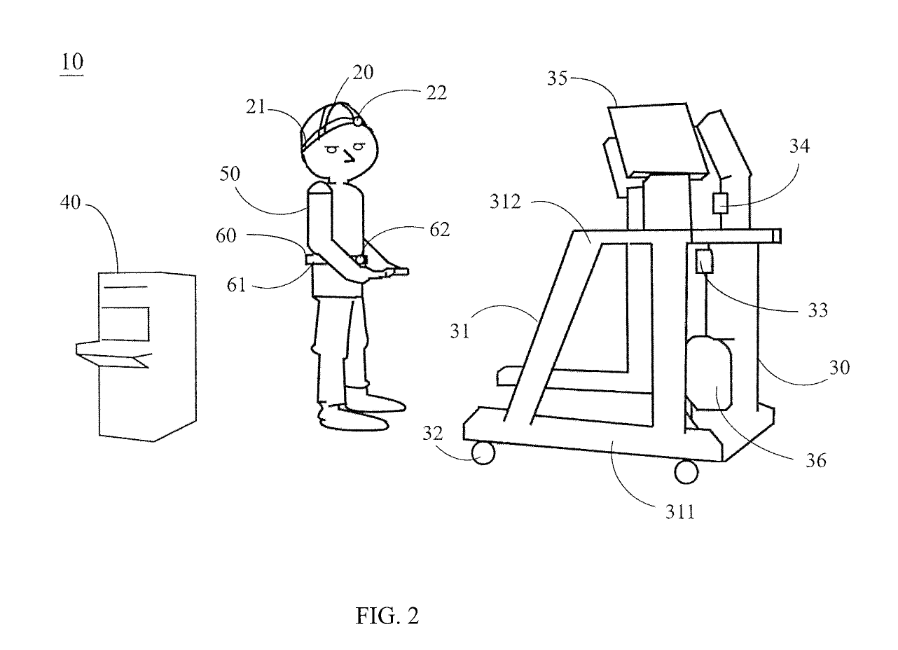

Please referring to FIGS. 1 and 2 of the invention drawing of the present application, the drawings are side view diagrams a dynamic assessment and rehabilitation system for vertigo patients of a preferred embodiment of the present application. As shown in the drawings, the dynamic assessment and rehabilitation system for vertigo patients 10 includes a first wearing unit 20, wherein the first wearing unit 20 includes a first wearing main body 21 ware on a head of a user 50 and at least a sensor 22 installed on the first wearing main body 21 and configured to sense at least a sensing signal of the user 50 and transfer the at least a sensing signal. In additional, the dynamic assessment and rehabilitation system for vertigo patients 10 further includes a moveable monitoring platform 30, wherein the moveable monitoring platform 30 includes a platform main body 31, a moving assistant assembly 32, a signal-receiving device 33, a distance sensor 34, at least a display device 35 and a control module 36, wherein the platform main body 31 includes two parts of a base 311 and a monitoring machine 312 connected above the base 311, and the moving assistant assembly 32 is connected to the lower surface of the base 311 to drive the moveable monitoring platform 30 to displace, in another aspect, the signal-receiving device 33, the distance sensor 34 and the at least a display device 35 are connected above the monitoring machine 312 and each has distinct function, for example, the signal-receiving device 33 is for receiving an image signal generated from when the user 50 is operating the system 10, and the distance sensor 34 is for measuring a monitored distance between the user 50 and the platform main body 31 to receive a distance signal, and the at least a display device 35 is for providing at least a display information. In additional, the control module 36 of the moveable monitoring platform 30 can be arranged at any position of the platform main body 30 and electrically connected to each of the signal-receiving device 33, the distance sensor 34, the moving assistant assembly 32 and the at least a display device 35, wherein the control module 36 receives and transfers the dynamic action signal, and the control module 36 further receives and analyzes the distance signal to drive the moving assistant assembly 32 to displace and controls the display device 35 to display a display information in accordance with a first instruction information. Finally, the dynamic assessment and rehabilitation system for vertigo patients 10 further includes a terminal modules 40 arranged at an any arranging point on the moveable monitoring platform 30 or within a signal-reachable range, and the terminal module 40 makes connections with the control module 36 and the at least a sensor 22, and the terminal module 40 receives the distance signal and the image signal transferred from the control module 36, and receives the at least a sensing signal coordinating the dynamic action signal to execute an assessment and recording of the physiological status of the user.

Wherein the at least a sensor 22 is for recording any activity signal generated from the head of the user 5 when the user 50 is operating the dynamic assessment and rehabilitation system for vertigo patients 10, and the signal can include the head position of the user 50 and the angular speed, acceleration or angular acceleration resulted from head-moving or head-turning. Especially, when the user is assessing the functions of the vestibular, the reference value of the assessment result is based on the amplitude and speed of the head-turning reaches a threshold value, in this way, after receiving the activity signal of the head of the user 50, the at least a sensor 22 can transfer the activity signal to the terminal module 40 for recording and analysis, and the terminal module 40 judges that the activity status reaches the threshold before providing the display information for the user 50 to test. Based on above principles, the at least a sensor signal includes an inclined angle signal of a head, an angular speed signal, an acceleration signal, an angular acceleration signal, a location signal of a head or any combination thereof, wherein the location signal of a head is a signal generated from a position change that the head of the use 50 moves around left to right or back and to forth, and the at least a sensor 22 can be any sensor device capable of sensing above any of the above sensing signals.

Wherein the at least a platform main body 31 includes the base 311 and the monitoring machine 312, and for making the at least a display 35 arranged on the monitoring machine 312 matching the height of sight light of most users, the monitoring machine 312 connects the base 311 by a way of forming in one piece, and the height is designed to be the average height of the user group. In additional, for making the platform main body 31 matching the sight line of every user for the best test demand, the base 311 and the monitoring machine 312 can be also connected by a telescopic element, and the telescopic element stretches along an extended direction perpendicular to a ground surface, and the monitoring machine 312 and the at least a display device 35 ca be adjusted corresponding to the height and the height of sight line of the user, therefore the accurateness for assessing the functions of a vestibular can be raised.

Wherein, for providing more pluralistic reference data to be the basis for users or treatment staffs judging the functions of a vestibular, the dynamic assessment and rehabilitation system for vertigo patients 10 includes the signal-receiving device 33 for receiving an image signal generated from when the user is operating the system 10, and when the user is executing a dynamic assessment or rehabilitation, the body stability of the user can be judged via receiving and analyzing the image signal, meanwhile, via the control module 36, the image signal can be recorded and analyzed in the terminal module 40 to be one of the reference data for judging the functions of a vestibular. Based on aforesaid description, the signal-receiving deice 33 can be an image capturing module, by receiving and analyzing the image signal of body-swing when the user is executing a dynamic assessment to achieve an object of providing more pluralistic reference data for judging the functions of a vestibular.

Moreover, for realizing the body stability of the user when the user is operating the system 10, the present application can further provide at least a second wearing unit 60 capable of projecting a displacement signal received by the signal-receiving device 33, and the displacement signal can be a signal straightly projected and can displace along with the movement of the body, and after keeping measuring the displacement signal for a period, the displace signal can be used for representing the condition of body-swing when the user is operating the system 10. The at least a second wearing unit 60 includes a second wearing main body 61 and a message-emitting device 62, wherein the second wearing main body is ware on the trunk of the user 50, and the message-emitting device 62 is arranged on the second wearing main body 61 and capable of projecting a displacement signal to the signal-receiving device 33. Wherein the trunk can be the chest, abdomen, pelvis and buttock of a human body, and by wearing the at least a second wearing unit 60 on the trunk, the signal-receiving device 33 can really receive the body-swing condition and transfer that to the terminal module 40 via the control module to be recorded and analyzed, and when the user 50 wears multiple second wearing units 60 on the different parts thereof, the terminal unit can receive clearer body-swing message. In additional, the signal-receiving device 33 directly receives the dynamic message differs from the signal-receiving device 33 receives the positioning message from the second wearing unit 60 by that it's unnecessary to extra process the positioning signal but directly recording the received data and using the data to be a reference data for judging, and the image signal needs a further signal analysis before executing the record of body-swing, however, both of the above methods are effective for providing the reference data of body-swing. Based on above content, the displacement signal includes a light signal, a sound signal, a WiFi signal or a Bluetooth signal.

Continually, the message-emitting device 62 of the present application can not only provide the displacement signal to the signal-receiving device 33 to be a reference data of body-swing condition, but using measuring and transferring an inertial signal to be another reference data of body-swing condition, and it is unnecessary for providing a corresponding device to the inertial signal, and the inertial signal is a signal source not needing further process, so that the inertial can be directly transferred to the terminal module 40 to be recorded and to be a reference data for judging. Based on above description, the inertial signal can be an angular speed signal, an acceleration signal, an angular acceleration signal, a signal for sensing magnetic field position or the combination thereof.

Wherein the moveable monitoring platform 30 has a distance sensor 34 for ensuring the user 50 keeping a monitoring distance with the moveable monitoring platform 30 when the user executes tests relating to the functions of the vestibular in a dynamic status. The moveable monitoring platform 30 can immediately measure the distance between the apparatus and the user 50 by utilizing the distance sensor 34, and a distance signal can be achieved and then transferred to the control module 36 to be analyzed and calculated before driving the moving assistant assembly 32 to displace, accordingly the moveable monitoring platform 30 can move corresponding to the moving speed of the user 50 to achieve the object of keeping a fixed monitoring distance between the moveable monitoring platform 30 and the user 50. However, the distance sensor 34 is not limited to be a specific type, and based on the theory and spirit of the present application, any technology regarding measuring the distance with a user is included in the claims of the present application.

Wherein the present application provides at least a display device 35 to display at least a display information to the user 50 to execute the test relating to the function assessment of vestibular. The number of the display device 35 can be only one to be disposed in front of the user 50 for providing instruction to the user 50 to execute corresponding responding actions, or two or more than two display devices 35 can be provided, wherein a tested angle can be the included angle formed the lines between the display device 35 at left side and the user and the display device 35 at right side and the user, respectively. The display devices 35 can take turns displaying information to promote the user turning the head thereof and execute reacting actions corresponding to the display information, or one or more than one display devices 35 with larger sizes are disposed to provide information sources more complicated in the way of a virtual scene, therefore assessing the vestibular condition of the user 50 when the user is observing the messages of daily life. Based on the above description, the display information of the present application can be a text information, an image information or a virtual scene information.

Wherein, for effectively integrating the distance signals, image signals and displacement signals received by the signal-receiving device 33 and the distance sensor 34, respectively, and controlling the moving assistant assembly 32 and the at least a display device 35 to operate, the present application provides a control module 36 to control all messages received and transferred by the moveable monitoring platform. Accordingly, the control module 36 is configured to receive all signals received by the signal-receiving device 33 and the distance sensor 40, transfer needed messages to the terminal module 40 to be analyzed, receive the first instruction message transferred from the terminal module 40 and drive the moving assistant assembly 32 and the at least a display device 35 in accordance with the distance signal and the first instruction message.

Continually, in exception of receiving the first instruction message transferred from the terminal module 40, the control module 36 can be input with a second instruction message for driving the moving assistant assembly 32 to displace and control the display information of the at least a display device 35, wherein the second instruction message can be a fixed or a gradient moving rate or display frequency for the user executing assessment or rehabilitation training by following a predetermined schedule.

Continually, in exception of receiving an instruction message transferred from the terminal module 40 or pre-input from a user or treatment staff, the control module 36 can also self-execute signal analysis and process after receiving the distance signal and the image signal, and a third instruction signal is generated to drive the moving assistant assembly 32 to displace and control a display information displayed on the display device 35. When the control module 36 directly executes a signal process and drives and controls elements related to the monitoring platform 30, the time of signal-transferring between modules can be decreased, therefore the operation of the moveable monitoring platform 30 can be more instinctive to the user, and can immediately respond the operation condition to be adjusted.

Wherein, for executing assessment and recording the operation response in the duration when the user operates the dynamic assessment and rehabilitation system for vertigo patients 10, the dynamic assessment and rehabilitation system for vertigo patients 10 of the present application includes a terminal module 40 for analyzing, judging and recording the data when the user is operating the system 10, and the terminal module 40 can be directly disposed on the moveable monitoring platform 30 or disposed at any disposing point within a signal-reachable range when the control module 36 is transferring signals. The kind of display information decided by the terminal module 40 to transfer to the user depends on the head-turning condition of the user. When the condition such as angle or angular speed of the head-turning meets the test standard for the vestibular functions, the terminal module 40 will release a display information related to the test topic. Instead, when the head-turning fails to meet the test standard for the vestibular functions, the user will not get the display information related to the test topic, or the display information will be replaced by that implies the user how to reach the test standard, or the terminal module 40 will provide the user a test topic with lower difficulty. Therefore the terminal module 40 receives the sensing signal transferred from the sensor 22, and analyzes and processes the sensing signal before providing the first instruction message to the control module 36 to drive the at least a display device 35 to display the display information, and the assessment of vestibular function of the user 50 can be executed. Wherein the connection between the terminal module 40, the control module 30 and/or the at least a sensor 22 can be electric connection or wireless communication connection.

Continually, in exception of the turning condition of the user 50, when the user 50 is operating the system 10, the terminal module 40 also assesses the vestibular functions of the user 50 in accordance with the response of the display information and the limb stability when the user 50 is taking an activity. Therefore a responding result generated from a response made by the user for the test topic will be transferred to the terminal module 40 by electric or man-made assist to be analyzed and processed, and then will be provided to drive the at least a display device 35 to update the display information in together with the sensing signal. In another aspect, after receiving the image signals or the positioning signals transferred from the control module 36, the terminal module 40 can not only record by coordinating the at least a sensor signal and assess the physiologic condition of the user, but provide an instruction message to the control module 36 to update the display information in together with the responding result of the sensor signal after the signal being analyzed and processed, therefore the object of assessing the vestibular functions of the user 50 under a dynamic activity by a more physiologic way.

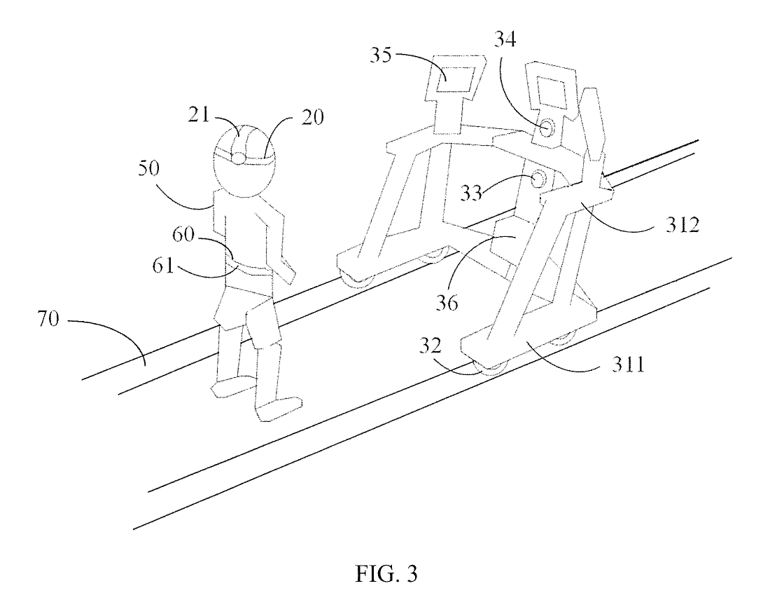

Please referring to FIG. 3 of the invention drawings of the present application, besides aforesaid elements, in another preferred embodiment, the dynamic assessment and rehabilitation system for vertigo patients 10 of the present application can further include a track 70 for the moveable monitoring platform 30 being disposed thereon to displace along a specific direction, meanwhile, the track 70 can also restrict the testing range of the user 50. By guiding the user to a predetermined direction to take a dynamic activity, the track 70 provides the user 50 another type of dynamic activities to execute the assessment and rehabilitation of vestibular functions.

In additional, in another preferred embodiment, for making the operation of the dynamic assessment and rehabilitation system for vertigo patients 10 can be more instinctive to the user, the present application further provides a training guiding module (not shown). When the user is in a training scene provided by the at least a display device 35, the training guiding module can prompt the user 50 the action corresponding to executing dynamic assessment or rehabilitation by another way, therefore the efficiency of the system executing assessment and rehabilitation of vestibular functions can be raised. Based on aforesaid principles, the training module can be a loudspeaker device, an earpiece device, a vibrating motivation device, a light-touching motivation device, a stroking motivation device, a tapping motivation device or an air-flow motivation device. By using the mentioned devices, the training guiding module provided by the present application can provide at least a prompt of training to the user 50 to execute training.

In summary, in accordance with aforesaid element configuration, the present application indeed provides a new dynamic assessment and rehabilitation system for vertigo patients, and the system not only provides the user a dynamic way to execute the assessment of vestibular functions to achieve the assessment result being closest to the life scene of the user, but provides multiple reference data to execute the assessment of vestibular functions by observing the limb stability of the user when the user is in activity condition, and records the related data in detail for providing the user or the treatment staff to refer to. In another aspect, the system can effectively and immediately adjust the training difficulty to be close to the rehabilitation demand of the user to execute an objective and accurate analysis, therefore achieving the object of raising entirety of efficiency.

Some elements, features of devices and the operation of the application method of the dynamic assessment and rehabilitation system for vertigo patients are further described below:

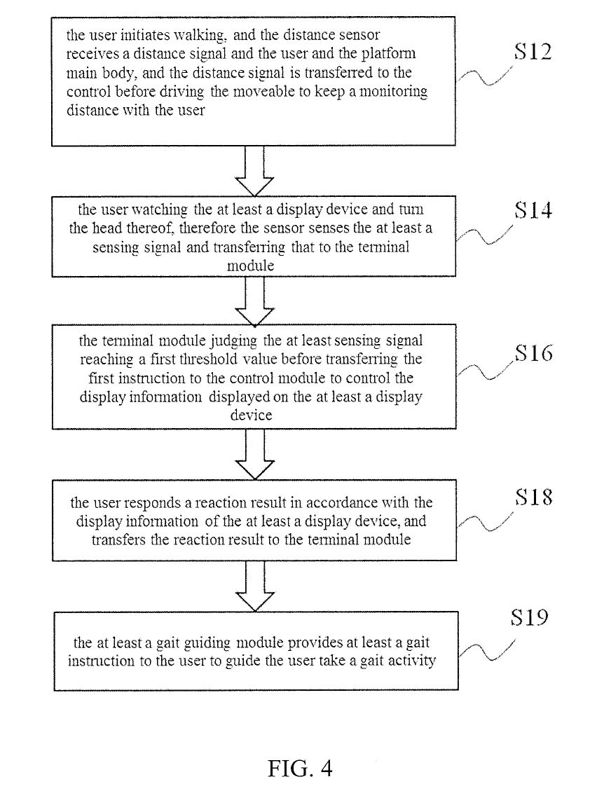

Please referring to FIG. 4, which is a method flow chart of the application method of the dynamic assessment and rehabilitation system for vertigo patients of an embodiment of the present application, as shown in the drawing, the application method of the dynamic assessment and rehabilitation system includes steps of:
Step S12: Keeping a monitoring distance between the moveable monitoring platform and the user while the user walking by receiving, executing and informing a distance signal;
Step S14: Sensing at least one sensing signal and transferring them to the terminal module while the user watching the at least one display device and turn the head thereof;
Step S16: Showing the display information on the at least one display device after the terminal module judging the at least one sensing signal reaching a first threshold value;
Step S18: Gaining a reaction result while the user reacting to the display information on the at least one display device;
Step S19: Repeating the step keeping a monitoring distance to step gaining a reaction result until the information gathered by the terminal module is much enough to assess the physiologic status of the user.

As the step S12 shown in the drawing, in the application method of the dynamic assessment and rehabilitation system of the present application, firstly the user starts to walk, and at this time the distance sensor 34 of the moveable monitoring platform 30 senses a distance signal between the user 50 and the platform main body 31, and after the distance signal is transferred to the control module 36 to be analyzed and processed, the control module 36 drives the moveable monitoring platform 30 to keep a monitoring distance with the user 50, accordingly ensuring the monitoring distance between the moveable monitoring platform 30 and the user is a fixed value when the user is in the process of assessing vestibular functions, therefore the stability and reliability of the reference data achieved from the application method can be ensured.

As the step S14 shown in the drawing, in the application method of the dynamic assessment and rehabilitation system of the present application, the display device 35 provides a prompt message to the user to ask the user take head-turning action to test the functions of the vestibular system of the user, wherein the prompt can be a direct text prompt or a two or more than two display devices alternating displaying in the test angle, or other prompt way capable of achieving the same effect for expecting the angle and rate of the head-turning of the user can reach an intensity enough for executing test of the vestibular system, meanwhile, the sensor 22 installed on the head of the user 50 can sense the at least a sensing signal representing the intensity of the head-turning of the user 50, and the sensing signal is transferred to the terminal module 40, wherein the type of the at least a sensing signal includes an angle signal of a head, an angular speed signal, an acceleration signal, an angular acceleration signal or a position signal of a head.

As the step S16 shown in the drawing, when the at least a sensing signal is transferred to the terminal module 40, the terminal module 40 executes analysis and processing in accordance with the sensing signal and compares the signal to a first threshold value, and the terminal module 40 judges the at least a sensing signal reaching the first threshold value before transferring a first instruction message to the control module 36 to control the display information of the at least a display device 35, at this time the at least a display device 35 displays the content of the test topics to the user for testing, otherwise, if the terminal module 40 judges the at least a sensing signal failing to reach the first threshold value, the application method would return to step S14, and the at least a display device 35 keeps providing prompt message to the user 50 to help the head-turning of user 50 reach the intensity enough to execute a test to the vestibular system.

Wherein the aforesaid display information includes a text information, an image information or a virtual scene information, and wherein the first threshold value includes: the angular speed of the head-turning of the user being between 30 degrees/second and 550 degrees/second; the displacement distance of the head being between 10 cm and 50 cm; the extent of the turning angle of the head being between 16 degrees and 60 degrees; or any combination thereof, wherein the displacement distance of the head includes laterally horizontal displacement or a horizontal displacement back to front, and the direction of the head-turning includes horizontally axial turning or vertically axial turning.

As the step S18 and S19 shown in the drawing, the user 50 answer the display information displayed on the at least a display device 35, that is, executing a test of vestibular functions, and a responding result is generated, and the responding result is transferred to the terminal module 40 via artificial help or electric answering to be judged. When the answering result is correct, the application method is back to step S12 to keep making the user 50 turn the head thereof in a dynamic condition and answer the testing content displayed on the at least a display device 35. Meanwhile, the application method provides different type or harder test contents to the user for testing until the terminal module 40 receives the responding judged as a wrong answer, at this time the information content gathered by the terminal module can be judged to be qualified to assess the physiologic condition of the user 50, and the treatment staff executes the assessment of vestibular functions of the user 50 by using the application method, therefore finishing this time of the dynamic assessment application method.

In a preferred embodiment, the moveable monitoring platform 30 is arranged in front of the user 50 wearing the first wearing unit 20 in advance and kept a predetermined monitoring distance with the user 50, in this way, in the beginning of the user taking a dynamic activity, the moveable monitoring platform 30 can displace in association therewith and give the user 50 prompt messages to execute head-turning and the step of answering, therefore the entirety of the dynamic assessment can be accelerated.

In additional, in another preferred embodiment, the moveable monitoring platform 30 can have an signal-receiving device 33 therein to receive an image signal resulted from that the user 50 operates the system, and the image signal is transferred to the terminal module 40 via the control module 36 to be recorded, and another objective fact can be a reference to the dynamic assessment result of the user 50, and the body stability when the user executes dynamic assessment or rehabilitation can be judged. Based on aforesaid content, the image-receiving device 33 can be an image-capturing module receiving and analyzing the image signal of body-swing of the user to achieve the object providing more pluralistic of reference data of vestibular functions.

Continually, in the preferred embodiment, a second wearing unit 60 can be provided for the user to wear, the second wearing 60 is capable of projecting a displacement signal received by the signal-receiving device 33, and the displacement signal can be a signal straightly projected and can displace along with the movement of the body, and after keeping measuring the displacement signal for a period, the displace signal can be used for representing the condition of body-swing when the user is operating the system 10. What's more, when the user wears multiple of the second wearing unit 60 on the different parts thereof, the message of body-swing received by the terminal module 40 can be clearer. Based on above content, the displacement signal includes a light signal, a sound signal, a WiFi signal or a Bluetooth signal.

Furthermore, in exception of providing the displacement signal to the signal-receiving device 33 to be the reference data of body-swing, the signal-emitting device 62 can additionally measure and transfer an inertial signal to be the reference data of body-swing, and the inertial signal is a signal source not needing further process, so that the inertial can be directly transferred to the terminal module 40 to be recorded and to be a reference data for judging, wherein the inertial signal can be an angular speed signal, an acceleration signal, an angular acceleration signal, a signal for sensing magnetic field position or the combination thereof.

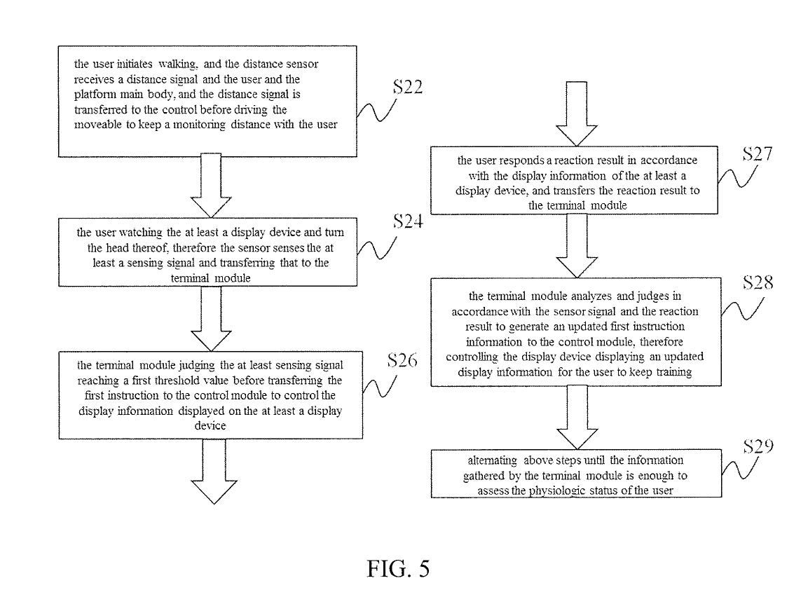

Please referring to FIG. 5, which is a method flow chart of the application method of the dynamic assessment and rehabilitation system for vertigo patients of another embodiment of the present application, as shown in the drawing, the application method of the dynamic assessment and rehabilitation system includes steps of:
Step S22: Keeping a monitoring distance between the moveable monitoring platform and the user while the user walking by receiving, executing and informing a distance signal;
Step S24: Sensing at least one sensing signal and transferring them to the terminal module while the user watching the at least one display device and turn the head thereof;
Step S26: Showing the display information on the at least one display device after the terminal module judging the at least one sensing signal reaching a first threshold value;
Step S27: Gaining a reaction result while the user reacting to the display information on the at least one display device;
Step S28: Analyzing and judging the sensor signal and the reaction result to generate an updated first instruction information, and refreshing the display information according to the updated first instruction information for the user to keep training;
Step S29: Repeating the step keeping a monitoring distance to step Analyzing and judging the sensor signal and the reaction result until the operation condition of the user reach a target of the rehabilitation.

The application method of the dynamic assessment and rehabilitation system for vertigo patients of another embodiment of the embodiment differs from that of the prior embodiment by that in the embodiment the terminal module 40 executes a composite analysis and judgement in accordance with the head-turning condition sensed form the sensor 22 and the responding result of that the user 50 answering the test, accordingly another new instruction message is formed and transferred to the control module 36 to control the at least a display device 35 to display an updated display information for the user 50 to keep training, and providing the user 50 a training mode meeting the current condition of the user 50 to achieve the object promoting vestibular functions.

As the step of step S22, similarly, in the application method of the dynamic assessment and rehabilitation system for vertigo patients of the embodiment, after the user 50 starts to walk, the distance sensor 34 senses a distance signal between the user 50 and the moveable monitoring platform 30, and a monitoring distance can be kept between the moveable monitoring platform 30 and the user 50 to ensure the stability and reliability of the reference data achieved from the application method can be ensured. Wherein the features of the elements used in the process and the way to processing signals is similar to that of the S12 of the drawing so it is unnecessary to go into details.

As the step of step S24, similarly, in the application method of the dynamic assessment and rehabilitation system for vertigo patients of the embodiment, at least a display device 35 is used to prompt the user 50 taking head-turning actions, and a sensing signal is measured and transferred to the terminal module via the control module 36 to be analyzed and processed. Wherein the features of the elements used in the process and the way to processing signals is similar to that of the S14 of the drawing so it is unnecessary to go into details.

As the step of step S26, similarly, in the application method of the dynamic assessment and rehabilitation system for vertigo patients of the embodiment, after judging whether the at least a sensing signal reaching a first threshold value, determining the sort of instruction message to be transferred to the control module 36 to control the display information of the at least a display device 35. Wherein the features of the elements used in the process and the way to processing signals is similar to that of the S16 of the drawing so it is unnecessary to go into details.

As the steps of step S26, S28 and S29, the application method of the dynamic assessment and rehabilitation system for vertigo patients of the embodiment asks the user 50 to answer the display information displayed on the at least a display device 35 for the training of vertigo lesion, in particular, being vestibular functions to form a responding result, and the responding result is transferred to the terminal module 40 via artificial help or electric answering to be judged to be one of the basis of the current training condition and the difficulty variety of following test topics during the training. When the answer is correct, the terminal module 40 identifies the condition of rehabilitation training of the user 50 is stable, and the current training difficulty can be remained for training or can be raised to a harder training level, on the contrary, when the answer form the user 50 is wrong, the system may lower the difficulty of the test to ensure the user 50 being able to take the rehabilitation training being closer to the practical condition thereof to purchase a best training efficiency. Meanwhile, the terminal module 40 also comprehensively assess the sensing signal previously achieved and analyzed to analyze, when the head-turning of the user 50 remains the intensity qualified to the demand of the training, the terminal module identifies the condition of rehabilitation training of the user 50 is stable, on the contrary, when the rate of head-turning of the user 50 starts to slow down and not qualified to the demand of the training, the system may lower the difficulty of the test to ensure the user 50 being able to take the rehabilitation training being closer to the practical condition thereof. In exception of aforesaid systematically auto-adjusting functions, the treatment staffs can also adjust in accordance with the training contents and intensity to execute the rehabilitation training mode most suitable to the user 50.

In a preferred embodiment, the moveable monitoring platform 30 is arranged in front of the user 50 wearing the first wearing unit 20 in advance and kept a predetermined monitoring distance with the user 50, in this way, in the beginning of the user taking a dynamic activity, the moveable monitoring platform 30 can displace in association therewith and give the user 50 prompt messages to execute head-turning and the step of answering, therefore the entirety of the dynamic assessment can be accelerated.

In a preferred embodiment during the rehabilitation, similarly, the moveable monitoring platform 30 can have an signal-receiving device 33 therein to receive an image signal resulted from that the user 50 operates the system, and the image signal is transferred to the terminal module 40 via the control module 36 to be recorded, and another objective fact can be a reference to the dynamic assessment result of the user 50, and the body stability when the user executes dynamic assessment or rehabilitation can be judged. Wherein the sorts of the signal-receiving device 33 and the image signal are similar to aforesaid embodiment so it is unnecessary to go into details.

Continually, in the preferred embodiment, a second wearing unit 60 can be provided for the user to wear, the second wearing 60 is capable of projecting a displacement signal received by the signal-receiving device 33 or the terminal module 40. Wherein the measuring mode of the displacement signal, the sorts of the signal and the possible varieties thereof are similar to aforesaid embodiment so it is unnecessary to go into details.

In the aforesaid preferred embodiment, in exception of adjusting the training intensity by using the aforesaid responding condition of the answer and the head-turning intensity, the terminal module 40 can also utilize the image signal or the displacement signal to be one of the judging foundations of the body stability of the user 50. When the body stability of the user 50 is high, the system may remain or strengthen training difficulty, on the contrary, when the body stability of the user 50 is not enough when the user is operating the system, the system may lower the difficulty of the test to ensure the user 50 being able to take the rehabilitation training being closer to the practical condition thereof to raise the efficiency of the training rehabilitation.

In additional, in another preferred embodiment, the application method of the dynamic assessment and rehabilitation system for vertigo patients of the present embodiment can further provide a training guiding module. The train guiding module can provide the user 50 a prompt message by ware on the user 50 or extra arranged, and the prompt message prompt the user 50 the currently proposed step and the needed activity intensity during rehabilitation, to help the user 50 execute a rehabilitation training in a condition that the eyes of the user is watching the display screen without extra distract to notice whether the actions are right, and the user 50 can smooth and accurately take every activity during a rehabilitation process. Based on aforesaid principles, the training guiding module can be a loudspeaker device, an earpiece device, a vibrating motivation device, a light-touching motivation device, a stroking motivation device, a tapping motivation device or an air-flow motivation device. The prompt message provided by the training guiding module includes a prompt sound prompt, a light-touching prompt, a stroke prompt, a tapping prompt or any combination thereof.

The following specific embodiments are used to describe the contents of organized technologies, features and results of the present application which can be implemented accordingly, but the protection range of the present application is not limited thereto.

### Embodiment 1

Exhibition of the Dynamic Assessment and Rehabilitation System for Vertigo Patients

A user wears a head wearing unit, and the head wearing unit includes a IMU (Inertiall Measurement Unit) 9-axis inertiall transferring sensor configured to sense the head-turning condition of the user, and the user wears wearing units capable of projecting laser light on the abdomen and buttock thereof, respectively, to project laser light to the moveable monitoring platform to observe the body-swing of the user during an examination process.

Regarding to the moveable monitoring platform, the platform disposes a platform main body connecting a base to a monitoring machine via a telescopic rod, and a set of wheels disposed below the base and driven by a motor, and a light-message receiver, ultrasound distance sensor and three display devices, and a control chip is provided to electrically connect all elements of the moveable monitoring platform, to manage all messages achieved in the process and make corresponding controls to the display device and the motor driving the wheels, therefore making the platform achieve the function including moving a fixed distance in association with the user and displaying answer information and prompt information to the user.

In additional, the dynamic assessment and rehabilitation system further connects a computer system capable of recording, analyzing and processing the signal of body-swing and head-turning of the user, and executing an assessment of vestibular functions and adjustment of training intensity by contributing the answer condition of the user answering the topics.

### Embodiment 2

Dynamic Assessment of Vestibular Functions of Vertigo Patients

A user stands in front of the moveable monitoring platform and starts to walk, and the moveable monitoring platform also starts to displace correspondingly to keep a distance with the user, and the body-swing of the user is measured by the wearing units respectively ware on the abdomen and the buttstock of the user and recorded by the computer system.

The user can be noticed to take head-turning when walking, and when the wearing device ware on the user's head senses the condition of head-turning of the user, the sensed signal can be transferred to the computer system to be analyzed and processed, and a topic can be displayed on the display screen for the user to answer when the sensed signal analyzed and processed is over a specific measuring threshold, and when the answer is correct, the testing condition gets harsher and harsher until the user answers a wrong answer, and by the difficulty of the passed answer made by the user, a treatment staff can observe the vertigo condition of the user and be helped to judge vestibular functions of the user.

### Embodiment 3

Dynamic Rehabilitation Training of Vestibular Functions of Vertigo Patients

A user stands in front of the moveable monitoring platform and starts to walk, and the moveable monitoring platform also starts to displace correspondingly to keep a distance with the user, and the body-swing of the user is measured by the wearing units respectively ware on the abdomen and the buttstock of the user and recorded by the computer system. The body-swing of the user can be one of the foundations of difficulty varieties of testing topic during the training process.

The user can be noticed to take head-turning when walking, and when the wearing device ware on the user's head senses the condition of head-turning of the user, the sensed signal can be transferred to the computer system to be analyzed and processed, and a topic can be displayed on the display screen for the user to answer when the sensed signal analyzed and processed is over a specific measuring threshold, and the computer system can comprehensively assess the answer condition of the user, the stability of body-swing and the rate of head-turning to make a foundation for adjusting the difficulty of the testing topic, and continually adjust the difficulty of the test and the training object of vestibular to raise the entirety of efficiency when the user executes rehabilitation training, meanwhile make a treatment staffs immediately predominate the rehabilitation condition of the user and execute effective adjustment.

Although the present application has been explained above, it is not the limitation of the range, the sequence in practice, the material in practice, or the method in practice. Any modification or decoration for present application is not detached from the spirit and the range of such.

What is claimed is:

1. A dynamic assessment and rehabilitation system for vertigo patients, comprising:
a first wearing unit comprising:
a first wearing main body, the first wearing main body is ware on a head of a user;
at least a sensor, the at least a sensor is installed on the first wearing main body to sense at least a sensing signal of the user and transfer the at least a sensing signal;
a moveable monitoring platform comprising:
a platform main body, the platform main body comprises a base and a monitoring machine connected on the base;
a moving assistant assembly, the moving assistant assembly is installed under the base;
a signal-receiving device, the signal-receiving device is arranged on the monitoring machine to receive an image signal generated from the user during operating the system;
a distance sensor, the distance sensor is arranged on the monitoring machine to measure a monitored distance between the user and the platform main body and to gain a distance signal;
at least one display device arranged on the monitoring machine to provide at least one display information;
a control module, arranged on the platform main body and electrically connected to each of the signal-receiving device, the distance sensor, the moving assistant assembly and the at least a display device, wherein the control module receives and transfers the dynamic action signal, also the control module further receives and analyzes the distance signal to drive the moving assistant assembly to displace, and controls display information of the at least one display device in accordance with a first instruction information; and
a terminal module, arranged at an any arranging point in the moveable monitoring platform or within a signal-reachable range, and connects with the control module and the at least one sensor, and the terminal module receives the image signal transferred from the control module, and then executes analyzing the signals before providing the first instruction information to the control module, and receives the at least one sensing signal coordinating the dynamic action signal to execute an assessment of the physiological status of the user.

2. The dynamic assessment and rehabilitation system for vertigo patients of claim 1, wherein the sensing signal comprises: an angle signal of a head, an angular speed signal of a head, an acceleration signal of a head, an angular acceleration signal of a head or a position signal of a head.

3. The dynamic assessment and rehabilitation system for vertigo patients of claim 1, wherein the base and the monitoring machine are formed in one piece to be connected or connected by a telescopic element, wherein the telescopic element stretches along an extended direction perpendicular to a ground surface.

4. The dynamic assessment and rehabilitation system for vertigo patients of claim 1, further comprising
a second wearing unit comprising:
a second wearing main body, the second wearing main body is ware on the trunk of the user; and
a signal-emitting device, the signal-emitting device is arranged on the second wearing main body to project a displacement signal to the signal-receiving device.

5. The dynamic assessment and rehabilitation system for vertigo patients of claim 4, wherein the displacement signal comprises a light signal, a sound signal, a WiFi signal or a Bluetooth signal.

6. The dynamic assessment and rehabilitation system for vertigo patients of claim 4, wherein the signal-receiving device is capable of measuring and transferring an inertial signal to the terminal module, wherein the inertial signal comprises an angular speed signal, an acceleration signal, an angular acceleration signal, a signal for sensing magnetic field position or the combination thereof.

7. The dynamic assessment and rehabilitation system for vertigo patients of claim 1, wherein the display information comprises a text information, an image information or a virtual scene information.

8. The dynamic assessment and rehabilitation system for vertigo patients of claim 1, wherein the control module is capable of being input with a second instruction message to drive the moving assistant assembly to displace and control a display information displayed on the display device.

9. The dynamic assessment and rehabilitation system for vertigo patients of claim 1, wherein the control module is capable of generating a third instruction information after receiving the distance signal by analyzing the signal by itself, therefore driving the moving assistant assembly to displace and controlling a display information of the at least one display device.

10. The dynamic assessment and rehabilitation system for vertigo patients of claim 1, wherein the communication of the terminal module and the control module or at least one sensor is established by an electrical connection or a wireless communication.

11. The dynamic assessment and rehabilitation system for vertigo patients of claim 1, further comprising a track, wherein the moveable monitoring platform is installed on the track to displace, and the patient executes assessment and rehabilitation in the track.

12. The dynamic assessment and rehabilitation system for vertigo patients of claim 1, further comprising a training guiding module, wherein the training guiding module comprises a loudspeaker device, an earpiece device, a vibrating motivation device, a light-touching motivation device, a stroking motivation device, a tapping motivation device or an air-flow motivation device.

13. A method of dynamically assessing the physiologic status of a vertigo patient by using the dynamic assessment and rehabilitation system of claim 2, comprising steps of:
Keeping a monitoring distance between the moveable monitoring platform and the user while the user walking by receiving, executing and informing a distance signal;
Sensing at least one sensing signal and transferring them to the terminal module while the user watching the at least one display device and turn the head thereof;
Showing the display information on the at least one display device after the terminal module judging the at least one sensing signal reaching a first threshold value;
Gaining a reaction result while the user reacting to the display information on the at least one display device; and
Repeating the step keeping a monitoring distance to step gaining a reaction result until the information gathered by the terminal module is enough to assess the physiologic status of the user.

14. The application method of claim 13, wherein the sensing signal comprises: an angle signal of a head, an angular speed signal of a head, an acceleration signal of a head, an angular acceleration signal of a head or a position signal of a head.

15. The application method of claim 13, wherein the first threshold value is the angular speed of the head turning of the user being between 30 degrees/second and 550 degrees/second, or is the head displacement being between 10 cm and 50 cm, or is the extent of the turning angle being between 15 degree and 60 degree or any combination thereof.

16. The application method of claim 13, wherein the displacement signal comprises a light signal, a sound signal, a WiFi signal or a Bluetooth signal.

17. The application method of claim 13, wherein the moveable monitoring platform is further arranged in front of the user wearing the first wearing unit in advance and kept the monitoring distance with the user before the step the user initiating walking.

18. The application method of claim 13, wherein the moveable monitoring platform is further arranged a signal-receiving device to receive an image signal generated from the user during operating the system, and transfers and records the image signal to the terminal module via the control module

19. The application method of claim 13, wherein the user wears at least one second wearing unit, and when the user starts to walk, the at least one second wearing unit projects a displacement signal to the signal-receiving device, and the displacement signal is transferred to the terminal module via the control module to be recorded.

20. The application method of claim 19, wherein the displacement signal comprises a light signal, a sound signal, a WiFi signal or a Bluetooth signal.

21. The application method of claim 19, wherein the at least a second wearing unit can directly measure and transfer signal comprising an angular speed signal, an acceleration signal, an angular acceleration signal, a sensing signal of magnetic field location or any combination thereof to the terminal module to be recorded.

22. An application method of a system using the dynamic assessment and rehabilitation system for vertigo patients of claim 1, configured to dynamically assess the physiologic status of a vertigo patient, comprising steps of:
Keeping a monitoring distance between the moveable monitoring platform and the user while the user walking by receiving, executing and informing a distance signal;
Sensing at least one sensing signal and transferring them to the terminal module while the user watching the at least one display device and turn the head thereof;
Showing the display information on the at least one display device after the tell final module judging the at least one sensing signal reaching a first threshold value;
Gaining a reaction result while the user reacting to the display information on the at least one display device;
Analyzing and judging the sensor signal and the reaction result to generate an updated first instruction information, and refreshing the display information according to the updated first instruction information for the user to keep training; and
Repeating the step keeping a monitoring distance to step Analyzing and judging the sensor signal and the reaction result until the operation condition of the user reach a target of the rehabilitation

23. The application method of claim 22, wherein the sensing signal comprises: an angle signal of a head, an angular speed signal of a head, an acceleration signal of a head, an angular acceleration signal of a head or a position signal of a head.

24. The application method of claim 22, wherein the first threshold value is the angular speed of the head turning of the user being between 30 degrees/second and 550 degrees/second, or is the head displacement being between 10 cm and 50 cm, or is the extent of the turning angle being between 15 degree and 60 degree or any combination thereof.

25. The application method of claim 22, wherein the display information comprises a text information, an image information or a virtual scene information.

26. The application method of claim 22, wherein the moveable monitoring platform is further arranged in front of the user wearing the first wearing unit in advance and kept the monitoring distance with the user before the step keeping a monitoring distance.

27. The application method of claim 22, wherein when the user starts to walk, the signal-receiving device receives an image signal when the user operates the system, and the image signal is transferred to the terminal module to be recorded to be a judging foundation for updating the first instruction message.

28. The application method of claim 22, wherein the user wears at least a second wearing unit, and when the user starts to walk, the at least one second wearing unit projects a displacement signal to the signal-receiving device, and the displacement signal is transferred to the terminal module via the control module to be recorded.

29. The application method of claim 28, wherein the displacement signal comprises a light signal, a sound signal, a WiFi signal or a Bluetooth signal.

30. The application method of claim 28, wherein the at least one second wearing unit can directly measure and transfer signal comprising an angular speed signal, an acceleration signal, an angular acceleration signal, a sensing signal of magnetic field location or any combination thereof to the terminal module to be recorded and to be a judging foundation for updating the first instruction message.

31. The application method of claim 22, wherein when the user executes a head-turning action, the system further provides a training guiding module providing a prompt message to help the user execute rehabilitation trainings.

32. The application method of claim 31, wherein the training guiding module comprises a loudspeaker device, an earpiece device, a vibrating motivation device, a light-touching motivation device, a stroking motivation device, a tapping motivation device or an air-flow motivation device.

33. The application method of claim 31, wherein the prompt message comprises a prompt sound prompt, a light-touching prompt, a stroke prompt, a tapping prompt or any combination thereof.
